(19) United States
(12) Patent Application Publication
(10) Pub. No.: US 2007/0292906 A1
(43) Pub. Date: Dec. 20, 2007

(54) ASSAY FOR MEASURING ACYLTRANSFERASE ACTIVITY

(75) Inventors: Dong Cheng, Furlong, PA (US); Luping Chen, Newtown, PA (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 11/762,796

(22) Filed: Jun. 14, 2007

Related U.S. Application Data
(60) Provisional application No. 60/813,554, filed on Jun. 14, 2006.

(51) Int. Cl. C12Q 1/48

(52) U.S. Cl. 435/15

(57) ABSTRACT

The subject matter disclosed and claimed herein relates to a high-throughput assay, and components thereof, for measuring the activity of acyltransferases. The high-throughput assay allows for rapid and accurate screening and identification of compounds that are modulators (e.g., inhibitors or activators) of acyltransferases, that may be used, for example, for the treatment of diabetes, diabetes-related disorders, obesity, cardiovascular disease, and other diseases or disorders attributed to acyltransferase activity.

This application claims priority to U.S. Provisional Application Ser. No. 60/813,554, filed on Jun. 14, 2006. The disclosures of this application and other publications referenced herein are hereby incorporated by reference in their entireties.

### FIELD OF THE INVENTION

The subject matter disclosed and claimed herein relates to a high-throughput assay, and components thereof, for measuring the activity of acyltransferases such as acyl-coenzyme A: diacylglycerol acyltransferase (hereinafter “DGAT”), acyl-coenzyme A: cholesterol acyltransferase (hereinafter “ACAT”), and acyl-coenzyme A: monoacylglycerol acyltransferase (hereinafter “MGAT”). The high-throughput assay allows for rapid and accurate screening and identification of compounds that are modulators (e.g., activators or inhibitors) of acyltransferases, that may be used, for example, for the treatment of diabetes, diabetes-related disorders, obesity, cardiovascular disease, and other diseases or disorders attributed to acyltransferase activity.

### BACKGROUND OF THE INVENTION

In mammals, neutral lipids, such as triacylglycerols (TAG), diacylglycerols (DAG) and cholesteryl esters (CE), are synthesized by acyl-coenzyme A: diacylglycerol acyltransferase (DGAT, EC 2.3.1.20), acyl-coenzyme A: monoacylglycerol acyltransferase (MGAT, EC 2.3.1.22), and acyl-coenzyme A: cholesterol acyltransferase (ACAT, EC 2.3.1.26), respectively. These acyltransferases share considerable similarities. They all possess multiple transmembrane domains, reside in the endoplasmic reticulum (ER), and catalyze the reaction involving the transfer of an acyl-moiety of acyl-coenzyme A to a hydrophobic substrate.

TAG is synthesized by two major pathways, the glycerol 3-phosphate pathway and the monoacylglycerol pathway (Bell, R. M., and Coleman, R. A. (1980) Annu Rev Biochem 49, 459-487), and is an important molecule for eukaryotic fuel storage. The glycerol 3-phosphate pathway is present in all tissues whereas the monoacylglycerol pathway is restricted to the enterocytes of the small intestine. The monoacylglycerol pathway is believed to be critical for the packaging of dietary fat into chylomicron lipoprotein particles (Levy, E. (1992) Can J Physiol Pharmacol 70, 413-419). DGAT is the common last step enzyme of both the glycerol 3-phosphate and monoacylglycerol pathways that catalyzes the terminal step in triacylglycerol synthesis by using diacylglycerol and fatty acyl-coenzyme A as substrates. To date, there are two known DGAT isozymes: DGAT1 and DGAT2. Interestingly, the two DGAT isozymes catalyze the same TAG synthesis reaction, yet they do not share substantial sequence similarity. There are also at least two isoforms of human DGAT1. One isoform has a histidine at position 129 and the other has a tyrosine at position 129. The DGAT1 isoform used by Applicants for the subject matter described herein has a tyrosine at position 129.

MGAT is the enzyme that initiates the monoacylglycerol pathway. In order for insoluble dietary fat, such as TAG, to be absorbed by the small intestine, dietary fat molecules must first be digested by pancreatic lipases into soluble free fatty acids and 2-monoacylglycerol. These products are quickly absorbed into enterocytes. MGAT uses these molecules as substrates to form DAG within minutes of entry into the lumen of the small intestine. DAG is further acylated by DGAT to re-form TAG. The newly formed TAG molecules are then packaged with other complex lipids such as cholesteryl ester, phospholipids and small amounts of protein, to form round lipoprotein particles called chylomicrons. Chylomicrons, 90% of which are comprised of TAG, are secreted into the lymph where they serve as a source of energy (Brindley, D. N., and Hubscher, G. (1965) Biochim Biophys Acta 106, 495-509). To date, the genes encoding three MGAT isozymes have been cloned (MGAT1, MGAT2, and MGAT3). MGAT2 and MGAT3 are highly expressed in the small intestine and may prove to have biological relevance in lipid metabolism.

ACAT enzyme activity is present in almost all mammalian cell types and tissues, with its highest activity found in macrophages (Brown M S, Goldstein J L, Annu. Rev. Biochem. 52:223-261(1983)), liver, small intestine, and adrenal glands (Chang T Y, Chang C C Y, Cheng D, Annu. Rev. Biochem. 66:613-638 (1997)). ACAT is believed to serve an important physiological role in foam cell formation, as the main composition of atherosclerotic plaque is cholesteryl ester (“CE”), an end product of the ACAT enzymatic reaction. ACAT is further believed to be essential for dietary cholesterol absorption in the small intestine. Therefore, modulating ACAT in these tissues is a potential therapeutic treatment for atherosclerosis and for reducing serum cholesterol.

Given the biological function(s) of acyltransferases, and the ACAT, DGAT and MGAT enzymes in particular, modulation of those enzymes may serve as a useful treatment for lipid/cholesterol/triglyceride-related metabolic disorders and diseases such as obesity and diabetes. Indeed, DGAT1 knockout mice, exhibited resistance to diet-induced obesity (Smith, S. J., Cases, S., Jensen, D. R., Chen, H. C., Sande, E., Tow, B., Sanan, D. A., Raber, J., Eckel, R. H., and Farese, R. V., Jr. (2000) Nat Genet 25, 87-90), and had improved insulin sensitivity (H. C. Chen, S. J. Smith, Z. Ladha, D. R. Jensen, L. D. Ferreira, L. K. Pulawa, J. G. McGuire, R. E. Pitas, R. H. Eckel and R. V. Farese, Jr., J. Clin Invest, 109 (2002) 1049-55).

Efficient assays to identify acyltransferase modulators have been difficult to develop. Conventional acyltransferase assays typically have low biological activity and are often contaminated by the products of other enzymatic reactions. Often times, the products of conventional assays are resolved by thin layer chromatography (TLC) analysis, which is not amenable to screening numerous activity modulators. Coleman et al., Meth. Enz., 1992; 209:98-104, describe a method for assaying DGAT function using organic solvents to extract TAG from isolated microsomes. However, the method of Coleman requires multiple extraction steps which prohibits the utilization of high-throughput screening of compounds that modulate DGAT activity. In view of the art-recognized shortcomings of assays used to measure acyltransferase activity, there is need for an accurate and reproducible high throughput assay for measuring acyltransferase activity (e.g. ACAT, DGAT, and MGAT) and agents that modulate such activity.

### SUMMARY OF THE INVENTION

Described and claimed herein is a method that overcomes the obstacles of traditional acyltransferase assays. The assay disclosed and claimed herein can be employed in a multi-well plate format to measure enzymatic activity and for high throughput screening of modulators of acyltransferase activity. In particular, Applicants' assay may be used to screen for modulators of ACAT, DGAT, and/or MGAT. One further component of the instant assay is the utilization of 2-monooleoylglycerol (hereinafter “MOG”) as a preferred assay substrate, over 1,2-dioleoylglycerol for measuring acyltransferase activity (e.g., DGAT and MGAT activities).

### DESCRIPTION OF THE FIGURES

FIG. 1. Panel A: Anti-FLAG Immunoblot of FLAG-tagged DGAT1 or MGAT; and Panel B: resolution of acyltransferase products revealed by TLC.

FIG. 2. Comparison of substrate efficiency determined in a TLC assay.

FIGS. 3A and 3B. Recombinant DGAT1 and control (Wild Type) membranes were used in Applicants' multi-well plate assays using 2-monooleoylglycerol (MOG) (Panel (A)) or 1,2-sn-dioleoylglyderol panel (B) as substrates.

FIG. 4. Recombinant DGAT1 (Panel (A)), MGAT3 (Panel (B)), or control membranes (Wild Type; Panel (C)) were assayed over time using MOG as the substrate.

FIG. 5. Various concentrations of MOG were evaluated in the multi-well plate assay using recombinant DGAT1 (Panel (A)) or using recombinant MGAT3 (Panel (B)).

FIG. 6. The DGAT1 inhibitor XP620 was used in the multi-well plate assay panel (A) and a TLC assay (Panel (B)), to compare the results obtained using a multi-well plate assay with the results obtained in a traditional low-throughput TLC assay.

FIG. 7. Statistical analysis of DGAT1 micro-plate assay.

FIG. 8. Statistical analysis of MGAT3 micro-plate assay.

### DETAILED DESCRIPTION OF THE INVENTION

The assay disclosed and claimed herein can be employed in a micro-plate format for high throughput screening for acyltransferase modulators. The high throughput assay has been validated against traditional TLC-based assays and proves to be an accurate and more rapid alternative to traditional TLC-based assays. Such a finding will facilitate high-throughput screening for acyltransferase modulators.

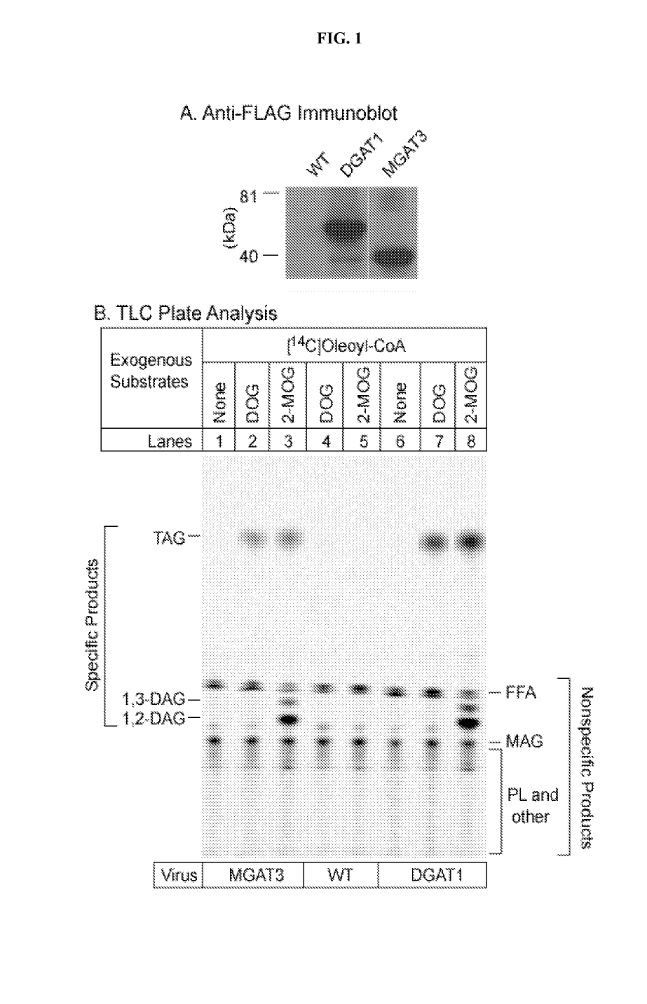

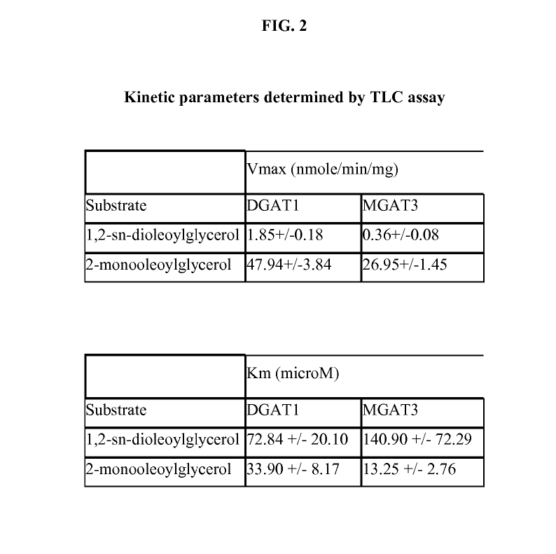

In general, the high-throughput assay described herein comprises: providing an acyltransferase enzyme; generating an enzyme-substrate composition by adding an enzyme substrate to said acyltransferase enzyme; delivering said composition to an assay vessel; initiating an enzymatic reaction in said assay vessel by delivering a labeled substrate to said composition; terminating said enzymatic reaction following an incubation period; and quantifying enzymatic activity by measuring label produced by said labeled substrate. Putative modulators can be added to the assay and differences in enzymatic activity (i.e., amount of label generated) between control wells and modulator-containing wells can be measured and compared. There are several preferred embodiments of the high-throughput assay described herein. For example the assay may further include ACAT1, ACAT2, MGAT1, MGAT2, MGAT3, DGAT1, or DGAT2 as the acyltransferase. Preferred acyltransferases are MGAT3 and DGAT1. The acyltransferases may be anchored to, or be a component of, membrane extracts. The acyltransferases may be an integral membrane protein or a synthetic enzyme anchored via a phosphatidyl bond. The assay further includes enzyme substrates such as 1,2-dioleoylglycerol and monooleoylglycerol (MOG). MOG is the most preferred substrate and has been found to be an excellent substrate for the assay disclosed herein (See FIGS. 1 and 2). The substrate employed in the assay may have a label conjugated thereto. Examples of labels that may be used include radiolabels such as [³H] or [¹⁴C]. Exemplary substrates include labeled long chain fatty acyl-coenzyme A such as [¹⁴C] oleoyl-CoA, [¹⁴C] palmitoyl-CoA, and [¹⁴C] stearoyl-CoA. In a preferred embodiment, the labeled substrate is [¹⁴C] oleoyl-CoA.

The assay may be conducted using any suitable assay vessel. Such vessels include single-volume vessels such as test tubes, Eppendorf tubes, or the like, as well as multiwell assay plates such as those having 3, 9, 12, 24, 48, 72, 96, 384, 1536 wells, and/or multi-well strips. Such assay vessels are commercially available (e.g., Perkin Elmer) and can be readily adapted for use in the assay described and claimed herein.

### EXAMPLES

The Examples described herein provide illustrative embodiments of the assay described and claimed herein. These examples are illustrative and would not be construed as limiting by those of ordinary skill in the art.

### Example 1

### Acyltransferase Assay Procedure

Spodoptera frugiperda (Sf9) insect cells were used to generate control membranes (i.e. membranes lacking exogenous acyltransferase(s)) and membranes containing recombinant acyltransferases. By way of example, DGAT1 and MGAT3 are described herein. Suitable Sf9 cell lines are commercially available from the American Type Culture Collection (“ATCC”). Control and DGAT/MGAT membranes were diluted in Buffer A (150 mM potassium phosphate, pH 7.4) at a final concentration 0.1 μg/μl, as measured using a Bradford assay with bovine serum albumin (“BSA”) as a reference standard. Following dilution in Buffer A, the membrane extracts were supplemented with various concentrations of MOG substrate in acetone, yielding a membrane/substrate mixture having a final substrate vehicle concentration of about 5%. Fifteen microliter (15 μl) aliquots of the membrane/substrate mixture (e.g, DGAT1 membranes/substrate, MGAT3 membranes/substrate, and control membranes/substrate) were then added to each well of 96 well PicoPlates (PerkinElmer).

One microliter (1 μl) aliquots of potential DGAT1 and/or MGAT3 inhibitor compounds (in DMSO) were then added to each well containing the membrane/substrate mixture. The membrane/substrate mixture was pre-incubated with the inhibitor compounds at room temperature for 10 minutes. Following pre-incubation, DGAT or MGAT reactions were initiated by the addition of 10 μl of 120 μM [¹⁴C]oleoyl-CoA, having a specific activity of 40,000 d.p.m./nmol, to each well containing the compounds, membranes, and substrates. After an incubation period at room temperature, (DGAT1-containing membranes incubated for 30 minutes and MGAT3-containing membranes incubated for 60 minutes), the reactions were terminated by the addition of 25 μl Buffer B (64.5% isopropanol, 16% heptane, 14% ethanol, 5.5% 2N NaOH) and 30 μl heptane. The 96 well plates were gently shaken for 15 minutes. Following shaking, 150 μl of MicroScint-O (PerkinElmer) were added to each well. The plates were gently shaken for an additional 15 minutes. The assay plates incubated (without shaking) for an additional 30 to 60 minutes to promote phase separation. The radioactivity in each well was then measured using a Packard Topcounter microplate scintillation counter. The relative acyltransferase activities are expressed as an arbitrary unit (counts per minute; “c.p.m.”) as determined from the data output generated by the scintillation counter.

### Example 2

### Screen of XP620 for DGAT1 Activity—TLC and Multiwell Formats

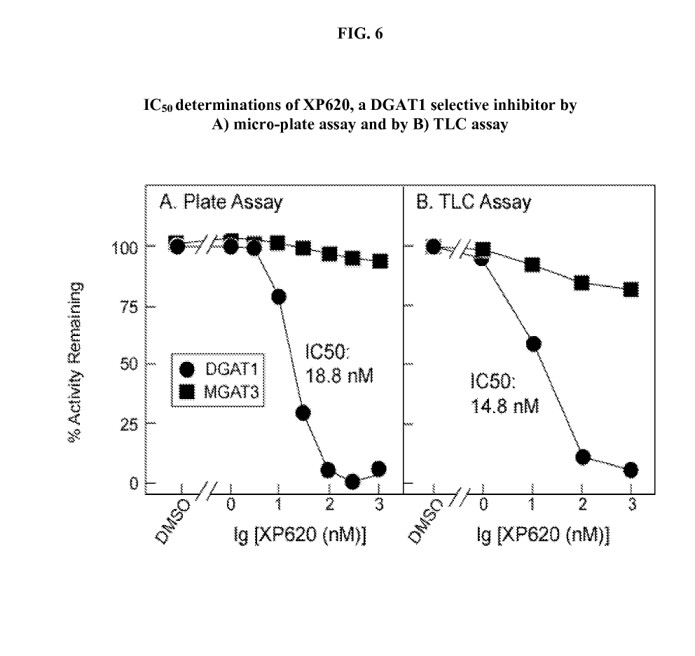

DGAT1 activity was measured in both a multiwell format (as described in Example 1) and in the traditional TLC assay using a known selective DGAT1 inhibitor, XP620. The data corresponding to this work is described in FIG. 6. XP620 was discovered and synthesized by Bristol-Myers Squibb Company, as reported in (Orland et al., Biochim Biophys Acta. 2005, Oct. 15;1737(1):76-82), which is incorporated herein by reference. Generally, because XP620 is selective for DGAT1, no inhibition of MGAT3 should be (and was not) observed.

Membranes containing either recombinant DGAT1, or MGAT3 were prepared as described in Example 1. XP620 was added to the respective membrane/substrate mixtures over a series of concentrations ranging from 0 nM of XP620 to about 1 μM XP620. The activity of the membrane-bound DGAT1 and MGAT3 was then measured using the micro-plate assay as described above (FIG. 6, Panel A), or using a traditional TLC assay (FIG. 6, Panel B).

For the TLC assay, 30 μg of protein in a membrane pellet of DGAT1 or MGAT3 recombinant membranes was mixed with 200 μl of 150 mM potassium phosphate buffer supplemented with 160 μM 2-monooleoylglycerol or 1,2-dioleoylglycerol (delivered by acetone, final vehicle concentration 5%, v/v). The reaction was initiated by adding 10 nmol [¹⁴C]oleoyl-CoA (having a specific activity 10,000 dpm/nmol, stock concentration: 1 nmol/μl). After incubating for 10 minutes at 37° C., the reactions were terminated by addition of 6 ml of chloroform/methanol (2:1, v/v). To facilitate phase separation, 1.2 ml of water was added to each well, mixed, and the plates allowed to incubate at room temperature for at least two hours. The aqueous phase was discarded. The organic phase containing the lipids was dried under nitrogen, resuspended in 100 μl chloroform, and spotted on ITLC-SA thin layer plates. Lipids were then separated by TLC using a solvent system comprising hexane: diethyl ether: acetic acid in a ration of 85:15:0.5, respectively, for about 20 minutes. Newly synthesized TAG and DAG bands were visualized and quantified using a STORM PhosphoImager. Specific DGAT or MGAT activities were calculated as nmol/min/mg protein.

For DGAT1 (FIG. 6, circles shown in Panel A), 1.5 μg membranes were used in the assay described in Example 1, and the reaction ran for 30 minutes. Likewise, for MGAT3 (FIG. 6, squares shown in Panel A), 1.5 μg membranes were used and the reaction ran for 60 minutes

The data described in FIG. 6 reflects that the IC₅₀ values for the assay disclosed and claimed herein, and the labor-intensive TLC assay are comparable (18.8 nM for the plate assay versus 14.8 nM for the TLC assay). As shown in FIG. 6, the MGAT3-containing membranes maintained near 100% activity when exposed to XP620, whereas DGAT1-containing membranes were sharply inhibited by XP620 in both the multiwell plate assay and the traditional TLC assay. The responsiveness of the system and the similarities in IC₅₀ values, reflects that the high-throughput multi-well plate assay provides an accurate, efficient, and high-throughput alternative to the time consuming TLC assay typically used to measure acyltransferase activity.

### Example 3

### Detection and Analysis of Recombinant Acyltransferases

Immunoblots were generated to verify proper expression of the nucleic acids encoding the recombinant acyltransferases used in Examples 1 and 2. The data from this set of experiments is reported in FIG. 1A. To facilitate detection of recombinant enzymes, a FLAG-epitope tag was fused in-frame at the amino termini of recombinant DGAT1 and/or MGAT3. The vectors encoding the FLAG-tagged human DGAT1 and human MGAT3 were expressed in Sf9 cells as described in Cheng et al., Biochem J. 2001 Nov. 1;359(Pt 3):707-14 and Cheng et al., J Biol Chem. 2003 Apr. 18;278(16):13611-4. As noted previously, the human DGAT1 used herein and reported in the Cheng et al., publications contain a tyrosine at position 129. Membrane extracts derived from wild type baculovirus infected Sf9 cells (WT), cells infected with recombinant human DGAT1 baculovirus (DGAT1), or with recombinant human MGAT3 baculovirus (MGAT3), were subjected to SDS-PAGE and immunoblot analysis by anti-FLAG IgG (available from Sigma). As shown in FIG. 1A, the Flag-tagged recombinant enzymes were expressed in the membranes.

The data shown in FIG. 1B correspond to a TLC plate assay and various substrates that could be used in the assay. Aliquots of membrane extracts (30 μg) derived from either wild type baculovirus infected Sf9 cells (WT) (lanes 4,5) infected with recombinant human DGAT1 baculovirus (DGAT1) (lanes 6-8), or with recombinant human MGAT3 baculovirus (MGAT3) (lanes 1-3), were subjected to enzyme reactions in Buffer A (150 mM potassium phosphate buffer, pH 7.4) with 50 μM [¹⁴C]oleoyl-CoA (40,000 d.p.m./nmol specific activity) as the acyl donor, and 100 μM 1,2-dioleoylglycerol (DOG), or 2-monooleoylglycerol (2-MOG) as the acyl acceptor (delivered by acetone, final vehicle concentration at 2%).

Assays were carried out for 10 minutes as described in (Cheng et al., Biochem J. 2001 Nov. 1;359 (Pt 3):707-14.). The products were resolved by TLC in a solvent comprising hexane: ether: acetic acid in a ration of 170:30:1, respectively. The products were visualized by a Phosphoimage exposure. The signals designated TAG (triacylglycerol),1,3-DAG, 1,2-DAG (diacylglycerol) are specific products, as they are specific to DGAT1 and MGAT3 recombinant proteins. The signals designated FFA (free fatty acids), MAG (monoacylglycerol) PL and Other (phospholipids and other products) are nonspecific products as they appear in WT control membranes.

The assay disclosed herein overcomes an art-recognized the hurdle in designing higher-through put assays for acyltransferase activity. That is, the assay allows identification of conditions that differentiate specific enzymatic products from non-specific products without engaging in the tedious, time consuming TLC procedure.

### Example 4

One of the discoveries made by Applicants and disclosed herein is that MOG serves as a superior substrate for acyltransferase assays because MOG is efficiently used by DGAT1 and MGAT3. The data supporting this conclusion are shown in FIG. 2. Briefly, substrate efficiency was determined from results obtained using a TLC acyltransferase assay. The substrate concentration titration was conducted with 1,2-sn-dioleoylglycerol and MOG in the TLC human DGAT1 and MGAT3 assays described in Example 3 and reported in FIG. 1B. The enzymatic products were quantified and the kinetic parameters Vₘₐₓ and Kₘ were derived using the Michaelis Menton Equation as applied by Prism® software. Assays were performed in duplicate and standard errors generated during the line fit are indicated. The analyses led to the conclusion that both DGAT1 and MGAT3 use MOG more efficiently, because the Vₘₐₓ value was more than 20-fold greater for DGAT1 and more than 50-fold greater for MGAT3 and the Kₘ value was significantly reduced for DGAT1 and MGAT3.

### Example 5

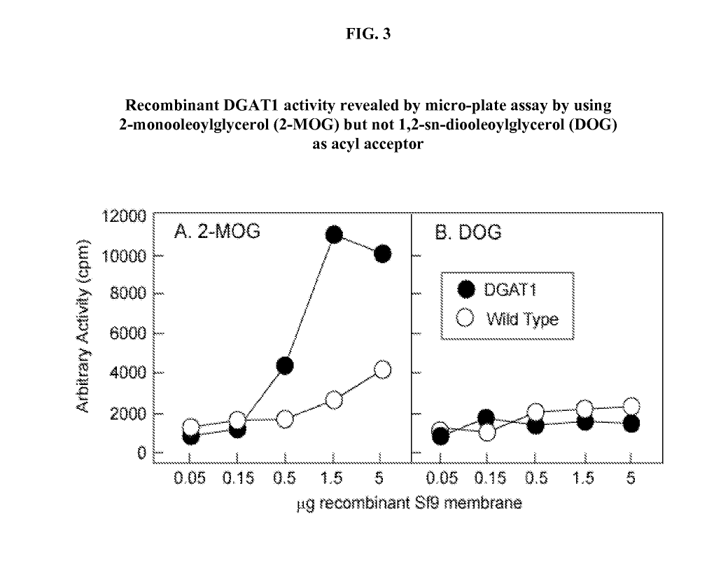

The increased efficiency and utility of MOG over DOG as the donor substrate was demonstrated in the multi-well plate assay. The data corresponding to this work is described in FIGS. 3A and B. The multi-well plate assay was performed essentially as described in Examples 1 and 2. Briefly, recombinant DGAT1 and control (Wild Type) membranes ranging from 0.05 μg to 5 μg (see x-axis of FIGS. 3A and 3B), were used. Either MOG (panel (A) in FIG. 3) or DOG (panel (B) in FIG. 3) were used as substrates. Thirty minute reactions were carried out at room temperature in duplicate. The mean values for TAG formation (represented by cpm on the Y-axis of FIG. 3) were plotted. The data demonstrates that MOG provides greater activity over the same membrane concentration when compared to DOG, in the same assay format. This finding suggests that use of MOG provides a more sensitive result when compared with DOG. When applying the current micro-plate assay, a careful titration is necessary for an optimal detection of specific signal associated with the recombinant acyltransferases. In this example, 1.5 μg protein of recombinant Sf9 DGAT1 is optimal, as it provides the biggest separation from the non-specific signal determined by the control membrane.

### Example 6

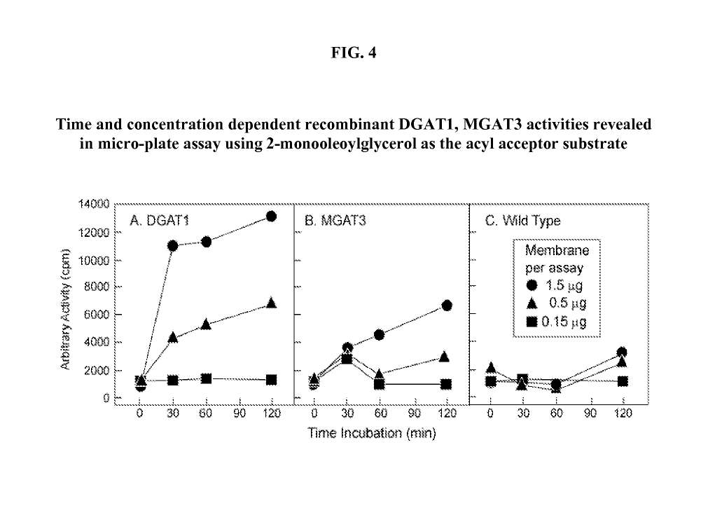

Additional assays were conducted to characterize the multiwell assay using MOG as the donor substrate. The data corresponding to this work is described in FIG. 4. Briefly, the multi-well plate assays, using MOG as the substrate, were set up and performed in duplicate as essentially described in Examples 1 and 2. In this series of experiments five time points (time 0, 30, 60, 90 and 120 minutes) were used to measure DGAT1, MGAT3 and wild type (control) acyltransferase activity. Overlaid on the time point component of this series of experiments, was the use of three different membrane concentrations (0.15 μg, 0.5 μg, and 1.5 μg) at each time point. This type of experiment allowed for evaluation of the differences in activity both in a time dependent manner and in a membrane concentration-dependent manner.

As described in the data (FIG. 4, Panels A, B and C), 0.15 μg of membrane provided very little activity in DGAT1, MGAT3, and control membranes at all time points. The intermediate membrane concentration (0.5 μg) yielded consistent activity for DGAT1 and presented a fairly linear slope over time, whereas the data for the 0.5 μg MGAT3 and control membranes was not as consistent. Finally, the 1.5 μg membrane concentration for DGAT1 and MGAT3 provided fairly robust activity levels. The activity level for DGAT1 reached near maximum after 30 minutes whereas MGAT3 showed a more linear slope for it peak activity. The results of this series of experiments suggests that 1.5 μg of membrane is an effective amount of membrane preparation to obtain a maximal response and can be obtained within 30 minutes or less.

### Example 7

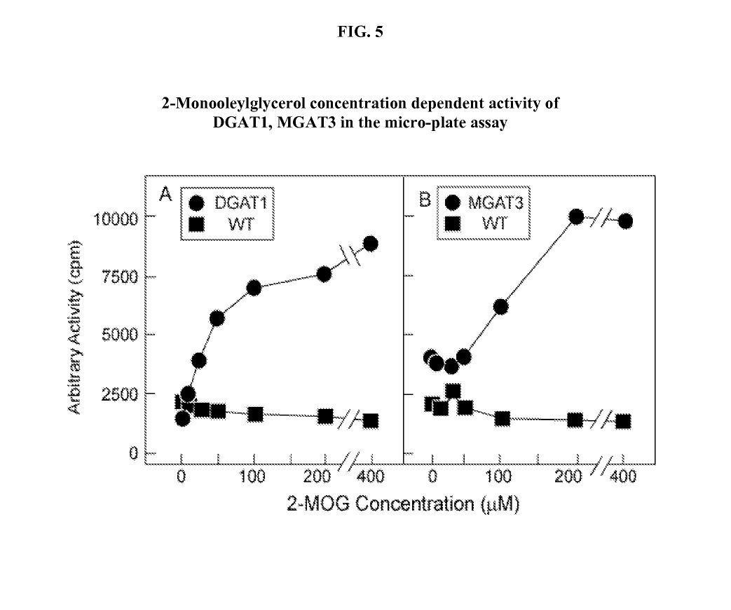

Additional assays were conducted to characterize acyltransferase activity in the multi-well plate assay. In this series of experiments, various concentrations of MOG were used and the activities of DGAT1 and MGAT3 were evaluated. These data are described in FIG. 5. Briefly, various concentrations of MOG were used in the multi-well plate assay described in Example 1 with either recombinant DGAT1 (FIG. 5, Panel (A)) or recombinant MGAT3 (FIG. 5, Panel (B)). Wild Type (WT) baculovirus infected Sf9 membranes were used as controls. The assays were conducted in duplicate and the mean activity values were plotted as shown in FIG. 5. This type of experiment further defines the optimal substrate (MOG) concentration to use in the multi-well-plate assay. The data reflect that, for DGAT1, the linear range is up to 50 μM, but saturated at above 100 μM and for MGAT3, the linear range is up to 200 μM, but saturated when above that level.

### Example 8

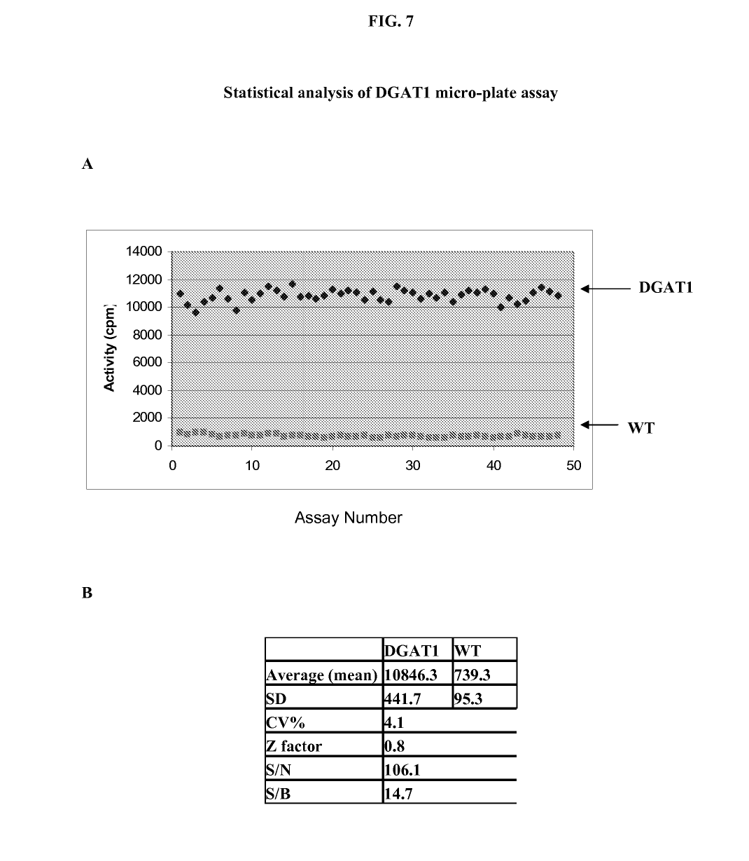

Statistical analyses were performed using DGAT1 activity as a test acyltransferase in a multi-well plate assay. The data from these experiments are described in FIGS. 7A and 7B. Forty-eight assays (as essentially described in Examples 1 and 2) were carried out in 96-well micro-plates containing 1.5 μg DGAT1 or control (WT) membranes, 200 μM MOG and 50 μM [¹⁴C]oleoyl-CoA, and the relative DGAT1 activity measured and reported. The signals from control wells were used to define the background level. The statistical parameters (Panel B) were derived using standard formulas. As shown from the data, the average maximum activity is 10,846+/−441 and the background signal was very low at 739.3+/−95.3. The Z factor was determined to be ˜0.8. These data reflect that the assay has a favorable signal/noise ratio which is indicative of a robust yet sensitive assay.

### Example 9

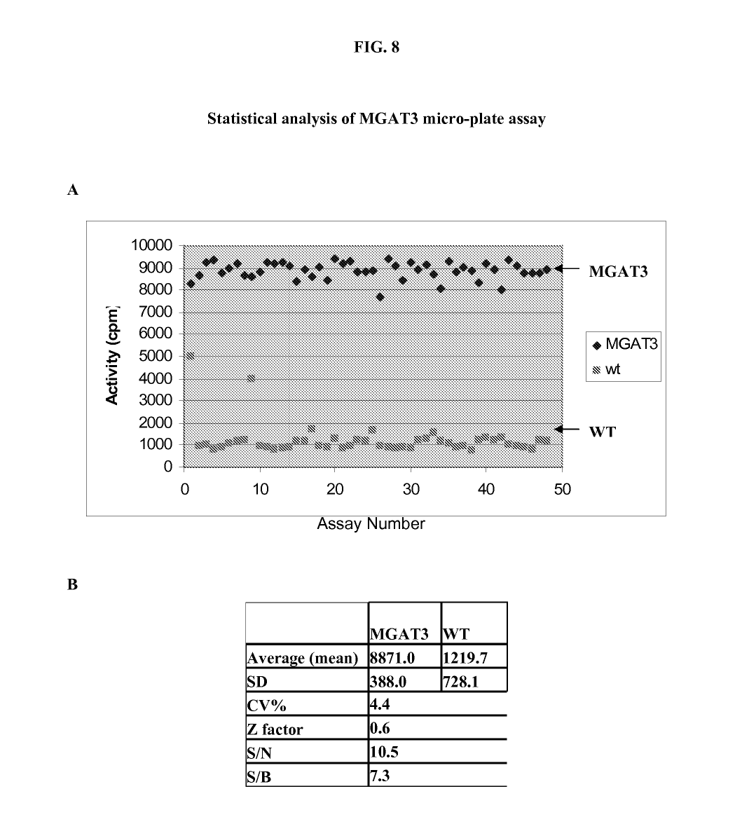

Statistical analyses were performed using MGAT3 activity as a test acyltransferase in the multi-well plate assay. The data from these experiments are described in FIGS. 8A and 8B. Forty-eight assays (as essentially described in Examples 1 and 2) were carried out in 96-well micro-plates containing 1.5 μg MGAT3 or control (WT) membranes, 200 μM MOG and 50 μM [¹⁴C]oleoyl-CoA and the relative MGAT3 activity measured and reported. The signals from control wells were used to define the background level. The statistical parameters (panel B) were derived using standard formulas. As shown from the data, the average maximum activity is 8871+/−388 and the background signal was very low at 1219.7+/−728. The Z factor was determined to be ˜0.6. These data reflect that the assay has a favorable signal/noise ratio which is indicative of a robust yet sensitive assay

The preceding examples are intended to be exemplary embodiments of the subject matter disclosed and claimed herein and are not intended to be limiting.

What is claimed is:

1. A high-throughput screening method for measuring enzyme activity comprising:
a) providing an acyltransferase enzyme;
b) generating an enzyme-substrate composition by adding an enzyme substrate to said acyltransferase enzyme;
c) delivering said enzyme-substrate composition to individual wells of a multiwell assay vessel;
d) initiating an enzymatic reaction in said individual wells by delivering a labeled substrate to said enzyme-substrate composition;
e) terminating said enzymatic reaction following an incubation period; and
f) quantifying enzymatic activity in the individual wells by measuring the amount of label produced from said labeled substrate following cleavage by said acyltransferase enzyme.

2. The method of claim 1, wherein said acyltransferase is an ACAT, a MGAT or a DGAT.

3. The method of claim 2, wherein ACAT, MGAT or DGAT are in membrane extracts.

4. The method of claim 2 wherein said acyltransferase is human ACAT1, ACAT2, DGAT1, or MGAT3.

5. The method of claim 1 wherein said assay provides a measure of acyltransferase activity that is within an order of magnitude of a traditional thin layer chromatography plate assay.

6. The method of claim 1, wherein said enzyme substrate is 2-monooleoylglycerol (MOG).

7. The method of claim 1, wherein said assay vessel is a 96, 384, or 1586 multiwell assay plate.

8. The method of claim 1, wherein said labeled substrate is radiolabeled and conjugated to coenzyme-A.

9. The method of claim 7, wherein the substrate is MOG.

10. The method of claim 1 wherein the membrane concentration is from between about 0.05 micrograms to about 5 micrograms.

11. The method of claim 10 wherein the membrane concentration is 1.5 micrograms.

12. The method of claim 1 wherein said enzymatic reaction in said multiwell assay vessel incubates for a period from less than 1 minute to about 120 minutes.

13. The method of claim 12 wherein said enzymatic reaction incubates for a period of about 30 minutes.

14. The method of claim 9 wherein the concentration of said MOG is greater than 0 micromolar but less than about 400 micromolar.

15. The method of claim 14 wherein said MOG concentration is between about 25 and 200 micromolar.

16. The method of claim 15 wherein said MOG concentration is about 100 micromolar.

17. A high-throughput screening method for identifying a modulator of acyltransferase activity comprising:
a) providing an acyltransferase enzyme;
b) generating an enzyme-substrate composition by adding an enzyme substrate to said acyltransferase enzyme;
c) delivering said enzyme-substrate composition to individual wells of a multiwell assay vessel;
d) delivering a control, or a putative modulator to individual wells of the multiwell assay vessel comprising the enzyme-substrate composition;
e) initiating an enzymatic reaction in said individual wells by delivering a labeled substrate to said enzyme-substrate composition;
f) terminating said enzymatic reaction following an incubation period;
g) quantifying enzymatic activity in the individual wells by measuring the amount of label produced; and
h) comparing the amount of label produced in those wells containing the control, with the amount of label produced in those wells containing the putative modulator wherein a decreased amount of label in the putative modulator-containing wells reflects inhibition of enzyme activity and an increased amount of label reflects activation of enzyme activity.

18. The method claim 17 wherein said substrate is MOG, said acyltransferase is DGAT1, and said control is XP620.

19. The method of claim 18 wherein said control has an IC₅₀ within an order of magnitude of the IC₅₀ obtained in a low-throughput TLC assay.

20. A high throughput method for measuring enzyme activity comprising:
a) expressing a recombinant acyltransferase in Spodoptera frugiperda (Sf9) or other insect cells;
b) obtaining membrane fractions from said cells comprising said recombinant acyltransferase;
c) diluting said membrane fractions;
d) adding MOG to said membrane fractions to produce a membrane-substrate composition;
e) adding a volume of said membrane-substrate composition to a multiwell assay plate;
f) adding a volume of a potential acyltransferase modulator to each well in said assay plate;
g) pre-incubating the assay plate comprising the membrane-substrate composition and acyltransferase modulator for about 10 minutes;
h) initiating an enzymatic reaction by addition of [¹⁴C]oleoyl-CoA to each well containing said modulator and membrane-substrate composition;
i) terminating said enzymatic reaction by the addition of a Buffer B comprising about 64.5% isopropanol, about 16% heptane, about 14% ethanol, and about 5.5% 2N NaOH, and heptane;
j) providing shaking to said multi-well assay for about 15 minutes;
k) adding scintillation fluid to each well in said multiwell plate;
l) providing shaking to said multiwell plates for about 15 minutes;
m) incubating said multiwell plates without shaking for about 30 to about 60 minutes;
n) measuring the radioactivity present on said multiwell plate using a microplate scintillation counter; and
o) determining the relative acyltransferase activity present in the multiwell plate.
